(19) United States
(12) Patent Application Publication
(10) Pub. No.: US 2008/0085560 A1
(43) Pub. Date: Apr. 10, 2008

(54) Lysing Reagent For Simultaneous Enumeration Of Different Types Of Blood Cells In A Blood Sample

(76) Inventor: Bjorn Ekberg, Malmo (SE)

Correspondence Address: VOLENTINE & WHITT PLLC, ONE FREEDOM SQUARE, 11951 FREEDOM DRIVE SUITE 1260, RESTON, VA, 20190, US

(21) Appl. No.: 11/665,753

(22) PCT Filed: Oct. 20, 2005
(86) PCT No.: PCT/DK05/00681

§ 371 Date: Jun. 1, 2007

(30) Foreign Application Priority Data
Oct. 20, 2004 (DK) PA 2004 01609

(51) Int. Cl. G01N 33/48; G01N 37/00

(52) U.S. Cl. 436/17; 436/63

(57) ABSTRACT

A combined diluent and lysing reagent for simultaneously counting or analysis of various blood cells such as thrombocytes, leukocytes and leukocyte subpopulations, as well as analysis of hemoglobin. The reagent comprises a hemoglobin stabilizing/methemoglobin ligand compound selected from the group consisting of imidazole, imidazole derivatives, and combinations thereof, at least two quaternary ammonium salts, 1,3-dimethylurea and/or salts thereof; and an organic buffer. Accordingly, the reagent can be used in a method for determining the content of blood cells in a blood sample comprising: mixing a blood sample with a reagent according to the invention; and analyzing the content of the blood sample by employment of the Coulter principle and optionally an spectrophotometric analysis of the hemoglobin content.

### TECHNICAL FIELD

The present invention relates to a reagent for use in the simultaneous automatic electronic enumeration and volumetric discrimination, for example by Coulter counting, of different types of blood cells, such as leukocytes, thrombocytes, etc, in blood. Further, the present invention relates to a Coulter (impedance) counting apparatus containing the reagent, for example a Coulter counting apparatus with a disposable cartridge for characterizing cells suspended in a liquid, especially a self-contained disposable cartridge for single-use analysis, such as for single-use analysis of a small quantity of whole blood.

### BACKGROUND OF THE INVENTION

Information on the content of leukocytes (white blood cells), their subpopulations and thrombocytes (platelets) is an important tool for the physician in order to diagnose different diseases and monitor treatment. Furthermore, the concentration of hemoglobin, directly related to the number of erythrocytes, in the blood sample is also of great importance.

Thus, much effort has over the years been devoted to the development of automated blood cell counting systems. Automated blood cell counting systems can be divided into two major groups: those relying on the impedance cell sizing principle (equal to the Coulter principle) and those relying on the flow cytometry principle. Examples of such automated systems are Coulter AcT Diff based on impedance cell sizing and Bayer ADVIA 120 based on flow cytometry. Both principles can be combined with spectrophotometric techniques for analysis of additional components in the blood, such as hemoglobin.

U.S. Pat. No. 4,962,038 disclose a reagent system containing a blood diluent and a lysing agent for routine enumeration of traditional hemogram values and the determination of leukocytes. The blood diluent comprises procaine hydrochloride, ADA, chlorhexidine diacetate, 1-hydroxypyridine-2-thione, and dimethylolurea, and the lysing agent comprises at least one quaternary ammonium salt and an alkali metal cyanide.

U.S. Pat. No. 4,745,071 disclose a diluent agent, a lysing agent, and a detergent for differentiation between and enumeration of lymphocytes, neutrophils and leukocytes.

The blood diluent comprises 1,3-dimethylurea, 1-hydroxypyridine-2-thione, and ADA, the lysing agent comprises a single quaternary ammonium salt and potassium cyanide, and the detergent comprises, in addition to the components of the diluent, a wetting agent diazopon.

U.S. Pat. No. 5,227,304 disclose a diluent solution in combination with a detergent solution for routine enumeration of blood cells, a blood sample is divided into a red cell aliquot for enumeration of red blood cells (erythrocytes) together with platelets (thrombocytes), and a white cell aliquot for the enumeration of white blood cells (leukocytes) and hemoglobin. In addition to the described solutions a lysing solution (as decribed in U.S. Pat. No. 4,745,071, see above) is needed for enumeration of the white cell aliquot. The diluent solution comprises imidazole, dimethylurea, EDTA, sodium omadine, triadine-10, and triadine-3, and the detergent solution comprises triadine-10, sodum omadine, and Brij 35.

U.S. Pat. No. 5,958,781 discloses a method for determining hemoglobin and leukocytes with a diluent comprising EDTA, imidazole, and a mixture of hexahydro-1,3,5-tris(2-hydroxyethyl)-s-triazine and sodium 2-pyridinethiol-1-oxide and a lysing reagent comprising at least one quaternary ammonium salt and a hydroxylamine salt.

Traditionally, erythrocytes and thrombocytes are quantified in a blood sample after addition of a diluent solution, whereas a lysing agent is added to the same or a separate blood sample to release hemoglobin from the erythrocytes and permit the quantification of the leucocytes together with an analysis of the hemoglobin. A full analysis of both erythrocytes, thrombocytes, leucocytes and hemoglobin, thus requires several steps.

Accordingly, there is a need for developing a reagent that combines the function of a diluent and a lysing agent, and which simultaneously allows a manual or an automated enumeration of various blood cells such as, e.g., thrombocytes and leukocytes, together with an analysis of hemoglobin.

A simultaneously identification and quantification of leukocytes and thrombocytes using one combined reagent only, would require that a lysing agent can lyse the erythrocytes without significantly affecting the thrombocytes. Typically, known lysing agents severely decreases the size of the thrombocytes, whereby, in connection with quantification, signals generated by the remains of the thrombocytes overlap with the particle background noise level. Furthermore, it is believed that the background noise is to a large extent generated by debris stemming from erythrocyte membranes. The further the background noise level is reduced the more accurate the thrombocyte particles can be identified and quantified.

Known lysing agents typically contain a cyanide salt and due to the toxicity of hydrogen cyanide such agents must have an alkaline pH. At a pH above 8, the size of e.g. thrombocytes changes drastically. In order to obtain an accurate and precise simultaneously analysis of all the above mentioned blood cells, together with an analysis of hemoglobin, the pH-value of a combined reagent system must be close to neutral, i.e., well below 8. It has previously been described that a medium used for enumeration of thrombocytes must, in addition to having a suitable osmolality, have a pH in the range of 6.5-7.6 (U.S. Pat. No. 5,935,857, U.S. Pat. No. 4,745,071, U.S. Pat. No. 4,185,964) to preserve the cell-volume of the thrombocytes.

### SUMMARY OF THE INVENTION

The present invention relates to a reagent comprising a hemoglobin-stabilizing/methemoglobin ligand compound selected from the group consisting of imidazole, imidazole derivatives, and combinations thereof, at least two quaternary ammonium salts, 1,3-dimethylurea and/or salts thereof; and an organic buffer. The reagent is useful as a combined diluent and lysing agent allowing a simultaneously counting or analysis of various blood cells as well as analysis of hemoglobin.

Accordingly, the present invention provides a method for determining the content of blood cells in a blood sample comprising the steps of: mixing a blood sample with a reagent according to the invention; and analyzing the content of the blood sample by employment of the Coulter principle.

Further aspects of the invention, will become apparent from the following description.

### DETAILED DESCRIPTION OF THE INVENTION

It is an object of the present invention to provide a reagent that combines the function of a diluent and a lysing agent, and which simultaneously allows a manual or an automated counting or analysis of various blood cells and analysis of hemoglobin. Hereby enabling the integration of dilution and lysing of a blood sample in one single step.

It is a further object of the present invention to provide a reagent that preserves the volume of the thrombocytes and improves the decomposition of unwanted cell debris from red blood cells (erythrocytes), thereby reducing background noise and facilitating an improved and more precise analysis or counting of thrombocytes simultaneously with other blood cells.

The reagent according to the present invention contains an aqueous medium.

The inventors has surprisingly found, that the above-mentioned and other objects are fulfilled by a reagent comprising, preferably in addition to other compounds mentioned below, a hemoglobin-stabilizing/methemoglobin ligand compound selected from the group consisting of imidazole, imidazole derivatives, and combinations thereof, at least two quaternary ammonium salts, 1,3-dimethylurea and/or salts thereof; and an organic buffer.

It is an important advantage of the present invention that while the reagent affects various types of blood cells, e.g. the erythrocytes are substantially eliminated, the leukocytes decrease in volume and the thrombocytes also decrease in volume, the composition of the lysing reagent according to the invention is accurately controlled so that the decrease in volume of the thrombocytes is not significant for the ability of the thrombocytes to be counted in a Coulter counter. It has been verified that the identified parts of the remains of the thrombocytes were related to the original content of thrombocytes in the blood samples according to comparative methods. This is further described in example 1 below.

In order to correctly and easily quantify the content of hemoglobin in blood samples, this analyte has to be released from the erythrocytes (lysis) and preferably converted to a suitable complex. The complex has to be suitable from a spectrophotometric point-of-view. The major hemoglobin species present in the blood are transformed to the corresponding complexed methemoglobin in two steps. The first oxidizing step is achieved in the present invention by oxidation of deoxy-hemoglobin and oxy-hemoglobin by the action of the quaternary ammonium salts and oxygen. The oxidized hemoglobin species, i.e. methemoglobin, is in the second step according to the invention complexed with imidazole resulting in a methemoglobin-imidazole complex, which is stable and has well-defined spectrophotometric properties. The complex may thus be spectrophotometrically quantified using specific wavelengths, e.g., 545 and 880 nm. The former wavelength quantifying the complex and the latter one compensating for turbidity in the blood sample and other absorbances, which are not attributed to the content of hemoglobin.

Thus, the action of the quaternary ammonium salts is two-fold: lysing the erythrocytes and enabling the oxidation of hemoglobin. A draw back of using quaternary ammonium salts are their simultaneous lysing action on leukocytes and thrombocytes. This action is compensated by the presence of 1,3-dimethylurea in the present invention. The 1,3-dimethylurea helps the preservation of, in particular, the leukocytes and thrombocytes and the identification of the three subcell types lymphocytes, monocytes and granulocytes. Accordingly, the reagent according to the present invention improves preservation of the volume of thrombocytes thus improving ability to count thrombocytes, e.g. by lysing the thrombocytes to a smaller degree (cf. FIG. 1).

The erythrocyte cell membrane disintegrates completely during the action of the lysing reagent. Since the erythrocytes do not contain any cell organelles, small cell membrane debris is the only remains of this blood cell type. This debris contributes significantly to the generation of the background-noise of automatic blood cell counting systems based on impedance cell sizing.

It is an important advantage of the present invention that the reagent improves the decomposition of unwanted cell debris from red blood cells (erythrocytes), thereby reducing background noise and facilitating an improved and more precise analysis or counting of thrombocytes.

The hemoglobin stabilizing compound in the reagent is selected from the group consisting of imidazole, imidazole derivatives, and combinations thereof and may be in a concentration in the range from about 10 mmol/L to about 100 mmol/L, such as from about 25 mmol/L to about 75 mmol/L, from about 40 mmol/L to about 60 mmol/L, and preferably about 50 mmol/L. The hemoglobin stabilizing compound is preferably imidazole in a concentration of about 50 mmol/L.

The blood cells in a blood sample are lysed by at least two quaternary ammonium salts. The quaternary ammonium salts suitable for use in the present invention has the formula: 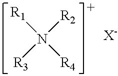
where R₁ is a long chain alkyl, alkenyl, or alkynyl radical having 10 to 18 carbon atoms, R₂, R₃, and R₄ are short chain alkyl, alkenyl or alkynyl radicals having 1 to 6 carbon atoms or just hydrogen, and X⁻ is a salt forming ion such as Cl⁻, Br⁻, I⁻, PO₄³⁻, CH₃SO₄⁻.

A combination of dodecyltrimethylammonium chloride and hexadecyltrimethyl-ammonium bromide is preferred, but other quaternary ammonium salts may be for example tetradecyltrimethylammonium bromide or hexadecyldimethylethylammonium bromide.

The total concentration of the at least two quaternary ammonium salts in the reagent may be in the range from about 1.0 mmol/L to about 10 mmol/L, such as from about 3.0 mmol/L to about 9.0 mmol/L, from about 4.5 mmol/L to about 8.5 mmol/L, and preferably about 6.5 mmol/L.

In a preferred embodiment of the invention the at least two quaternary ammonium salts is a combination of dodecyltrimethylammonium chloride and hexadecyltrimethyl-ammonium bromide in concentrations of about 5.6 mmol/L and about 0.9 mmol/L, respectively

As described above the 1,3-dimethylurea acts substantially as a cell stabilizing and antibacterial agent. The concentration of 1,3-dimethylurea and/or salts thereof in the reagent according to the present invention may be in the range from about 10 mmol/L to about 25 mmol/L, such as from about 12 mmol/L to about 23 mmol/L, from about 14 mmol/L to about 20 mmol/L, from about 16 mmol/L to about 18 mmol/L/L, and preferably about 17 mmol/L. In a preferred embodiment of the invention 1,3-dimethylurea is in a concentration of about 17 mmol/L.

The reagent according to the present invention further comprises an organic buffer, this organic buffer is a compound such as ADA (N-(2-acetamido)iminodiacetic acid), HEPES (2-(4(2-hydroxyethyl)-1-piperazine)ethanesulphonic acid), MOPS (3-(N-morpholino)propane sulphonic acid and PIPES (piperazine-N,N′-bis(2-ethane sulphonic acid) or combinations thereof and/or other organic buffers. Suitable concentrations of ADA, HEPES, MOPS and PIPES or combinations thereof in the reagent may be in the range from about 2 mmol/L to about 8 mmol/L, such as from about 4 mmol/L to about 6 mmol/L, from about 5 mmol/L to about 5.6 mmol/L, and preferably about 5.3 mmol/L. In a preferred embodiment of the invention the organic buffer is ADA with a concentration in the range from about 2 mmol/L to about 8 mmol/L, such as preferably from about 4 mmol/L to about 6 mmol/L, more preferably from about 5 mmol/L to about 5.6 mmol/L, and even more preferably about 5.3 mmol/L.

In addition to its buffering effect, ADA contributes to minimizing bacterial growth by its metal-ion chelating properties. Finally, ADA further assists the quaternary ammonium salts in reducing the red blood cells debris to a size minimizing interference with the quantification of thrombocyte particles close to the background noise as described above.

As mentioned above, there is a need to compensate for the action of the quaternary ammonium salts on the thrombocytes and leukocytes. In summary, the presence of 1,3-dimethylurea contributes to the preservation of the thrombocytes and the leukocyte subpopulations.

In a preferred embodiment, the reagent according to the present invention may further comprise procaine, procaine hydrochloride or other salts thereof. Procaine hydrochloride acts substantially as a cell stabilizing agent, and the concentration of procaine hydrochloride and/or salts thereof in the reagent may be in the range from about 0.2 mmol/L to about 1.6 mmol/L, such as from about 0.4 mmol/L to about 1.4 mmol/L, from about 0.6 mmol/L to about 1.2 mmol/L, from about 0.8 mmol/L to about 1.0 mmol/L, and preferably about 0.9 mmol/L.

Furthermore, to obtain a correct enumeration of the thrombocytes the cell volume must be preserved. At an elevated pH-value the size of the remains of the thrombocytes decrease further, whereby the thrombocytes generates signals that partly overlap the background noise signals, making the quantification of them more inaccurate. The pH of the reagent according to the present invention may therefore be adjusted to a suitable value in the range from about 6 to about 8, such as preferably in a range from about 6.5 to about 7.5, and more preferably about 7.1 with sulfuric acid or other suitable acids, such as hydrochloric acid, and phosphoric acid. A person skilled in the art will recognize that when a specific combination of substances in the reagent gives an pH-value below the desired pH-value, then an alkaline substance such as an inorganic base e.g. sodium hydroxide, may instead be used to adjust the pH-value. The osmolality may be adjusted to the range from about 200 mosmol/L to about 400 mosmol/L, such as preferably from about 250 mosmol/L to about 380 mosmol/L, more preferably from about 300 mosmol/L to about 350 mosmol/L, and even more preferably about 330 mosmol/L with an inorganic salt, such as sodium chloride, and/or sodium sulphate. In a preferred embodiment of the invention, the osmolality of the reagent is adjusted with sodium chloride and sodium sulphate. The blood sample may contain EDTA (ethylenediaminetetraacetic acid), salts thereof or any other suitable anti-coagulating compound such as heparin to prevent the blood sample from clotting.

In a specific embodiment of the invention, the reagent according to the invention comprises imidazole in a concentration of about 50 mmol/L; dodecyltrimethylammonium chloride in a concentration of about 5.6 mmol/L; hexadecyltrimethylammonium bromide in a concentration of about 0.9 mmol/L; 1,3-dimethylurea in a concentration of about 17 mmol/L; ADA (N-2-(acetamido)iminodiacetic acid) in a concentration of 5.3 mmol/L; and de-ionized water; wherein pH is adjusted to 7.1 with diluted sulfuric acid and the osmolality is adjusted with sodium chloride and sodium sulphate.

In another specific embodiment of the invention, the reagent according to the invention comprises imidazole in a concentration of about 50 mmol/L; dodecyltrimethylammonium chloride in a concentration of about 5.6 mmol/L; hexadecyltrimethylammonium bromide in a concentration of about 0.9 mmol/L; 1,3-dimethylurea in a concentration of about 17 mmol/L; ADA (N-2-(acetamido)iminodiacetic acid) in a concentration of 5.3 mmol/L; procaine hydrochloride in a concentration of 0.9 mmol/L; and de-ionized water; wherein pH is adjusted to 7.1 with diluted sulfuric acid and the osmolality is adjusted with sodium chloride and sodium sulphate.

Another aspect of the present invention is with regard to a method of determining the content of blood cells such as, e.g., thrombocytes, leucocytes, subpopulations of leucocytes, i.e. lymphocytes, monocytes, granulocytes, and hemoglobin, in a blood sample, e.g. an ex-vivo blood sample, the method comprising the steps of:
i) Mixing a blood sample, e.g. an ex-vivo blood sample, with a reagent according to the invention, and
ii) Analyzing the content of the blood sample, e.g., enumerating leukocytes, lymphocytes, monocytes, granulocytes and thrombocytes, by employment of the Coulter principle.

In a particular embodiment, the invention relates to a method of determining the content of thrombocytes, leukocytes and/or one or more leukocyte subpopulations such as, e.g., lymphocytes, monocytes and/or granulocytes. It is important to note that analysis of the various types of cells can be made simultaneously. The method according to the invention may furthermore comprise a step iii) Analyzing the content of the blood sample by spectrophotometric quantification of the hemoglobin content.

Furthermore, in the method according to the invention the ratio by volume between blood and reagent may be in the range from about 1:10,000 to about 1:200, such as from about 1:1,000 to about 1:300, from about 1:600 to about 1:400, from about 1:550 to about 1:450, and preferably about 1:500. The selected ratio depends on the concentrations of the compounds in the reagent, concentrations of blood cells and desired performance characteristics of the analysis.

For example, the reagent according to the present invention may be contained in a cartridge utilized in an apparatus for enumeration of cells in a blood sample, e.g. an ex-vivo blood sample, the cartridge comprising a housing with a mixing chamber and a collection chamber separated by a wall containing an orifice for passage of the cells between the mixing chamber and the collection chamber. Cell characterization means are provided for characterizing cells passing through the orifice. The apparatus comprises a docking station for removably receiving the cartridge, the docking station comprising connectors, e.g. electrical and/or fluid connectors, for operational connection with the cell characterization means when the cartridge is received in the docking station.

In a preferred embodiment of the invention, step i) of the method according to the invention, is performed in a cartridge suitable for use in an apparatus for enumeration of cells in a blood sample, in which cartridge the reagent is present in a holding chamber that is connected with a mixing chamber in which the reagent is mixed with the blood sample; and step ii) is performed by subsequent passage of the blood sample comprising cells through an orifice positioned between the mixing chamber and a collection chamber, whereby the content of the blood is analyzed by employment of the Coulter principle by cell characterization means.

Additionally, in a preferred embodiment, step iii) of the method according to the invention, is performed in a volume metering chamber connected to the collection chamber and adapted to spectrophotometric quantification.

In addition the reagent according to the present invention may be employed in a conventional apparatus for counting and determination of content of blood cells and hemoglobin in blood samples, e.g. ex-vivo blood samples.

The reagent may further be used as a coating to enhance the capillary effect of a tube or tube-like device. A coating comprising the reagent according to the present invention has shown to enhance the capillary effect of a hydrophobic tube or tube-like device. The reagent is coated onto a surface by evaporation of the reagent on the surface to be coated. Accordingly, a further aspect of the present invention is a coating comprising a reagent according to the invention.

A further aspect of the invention is the use of a reagent as defined above for the determination of the content of one or more cell types in a blood sample. In a particular embodiment, the invention relates to the use for the determination of thrombocytes, leukocytes and/or one or more leukocyte subpopulations such as, e.g., lymphocytes, monocytes and/or granulocytes in the same blood sample. It is important to note that the determination of the various types of cell can be made simultaneously. As discussed above, a specific embodiment of the invention relates to the use of the reagent for the determination of all cell types and subpopulations mentioned above. Moreover, another advantage is that the same reagent is suitable for use in the determination of the hemoglobin content in the blood sample, i.e. by use of only one single reagent relevant information of the blood content is obtained.

In this context the term “simultaneous determination” refers to a simultaneous determination in one analysis in the one and same blood sample. Accordingly, a blood sample is mixed with a reagent according to the invention and the various blood cell populations can be determined simultaneously in one analysis.

### BRIEF DESCRIPTION OF THE DRAWINGS

FIG. 1 is a histogram comparing the reagent according to the present invention and another single reagent system regarding their ability to count thrombocytes.

FIG. 2 schematically illustrates a cartridge accommodating the reagent according to the present invention for analysis of a blood sample.

FIG. 3 shows an apparatus for analysis of blood sample content utilizing a cartridge accommodating the reagent according to the present invention.

FIG. 4 shows a favorable histogram for counting leukocytes and leukocyte subpopulations, obtained by using the reagent described in Table 2.

FIG. 5 shows a favorable histogram for counting thrombocytes obtained by using the reagent described in Table 4.

FIG. 6 shows a favorable histogram for counting leukocytes and the leukocyte subpopulations, obtained by using the reagent described in Table 4; the histogram is the same as in FIG. 5, but with a different Y-axis.

### EXAMPLES

The composition of the presently most preferred embodiment of the inventive reagent is listed in Table 1:

TABLE 1

| Compound | Concentration range | |
|---|---|---|
| Imidazole | 48-52 | mmol/L |
| Dodecyltrimethylammonium chloride | 5.4-5.8 | mmol/L |
| Hexadecyltrimethylammonium bromide | 0.87-0.93 | mmol/L |
| 1,3-dimethylurea | 16.5-17.5 | mmol/L |
| Procaine hydrochloride | 0.87-0.93 | mmol/L |
| ADA (N-2-(acetamido)iminodiacetic acid) | 5.0-5.6 | mmol/L |
| Na₂SO₄ | 68-72 | mmol/L |
| NaCl | 40-42 | mmol/L |

| | |
|---|---|
| H₂SO₄, diluted | To obtain pH 7.1 |
| De-ionized water | To obtain 1 Liter |

The pH of the reagent is adjusted to 7.1 using diluted sulphuric acid, H₂SO₄.

### Example 1

### Correlation of Test Results Using a Reagent According to the Invention with Test Results Using a Method of Comparison

1 liter of reagent was prepared with a composition according to Table 2. The final osmolality of the prepared reagent was 330 mosmol/L.

TABLE 2

| Compound | Concentration | |
|---|---|---|
| Imidazole | 50 | mmol/L |
| Dodecyltrimethylammonium chloride | 5.6 | mmol/L |
| Hexadecyltrimethylammonium bromide | 0.9 | mmol/L |
| 1,3-dimethylurea | 17 | mmol/L |
| Procaine hydrochloride | 0.9 | mmol/L |
| ADA (N-2-(acetamido)iminodiacetic acid) | 5.3 | mmol/L |
| Na₂SO₄ | 70 | mmol/L |
| NaCl | 41 | mmol/L |
| H₂SO₄, diluted | 7 | mL (to obtain |
| 1/9 (vol./vol.) with water. | | pH 7.1) |

| | |
|---|---|
| De-ionized water | To obtain 1 liter |

Test results using a reagent according to the invention as described in table 2 were thereafter correlated with test results using a comparative system, the Beckman Coulter AcT Diff system. The test result includes quantification of leukocytes (including the three subpopulations lymphocytes, monocytes, and granulocytes), thrombocytes and hemoglobin. A total of 53 human whole blood samples were analyzed, where 3 μL of blood was mixed with 1.5 mL of reagent, which corresponds to a blood:reagent ratio by volume of 1:500. The tests were compared to tests performed on the Beckman Coulter AcT Diff system. The resulting correlations are listed in Table 3 for various concentration ranges of the content of leukocytes (including the three subpopulations lymphocytes, monocytes, and granulocytes), thrombocytes and hemoglobin in the blood samples. As can be seen from the table they demonstrate excellent correlation.

TABLE 3

| | | Linear correlation |
|---|---|---|
| Analyte | Concentration range | factor |
| Leukocytes | (1.4-68) · 10⁹/L | 1 |
| Lymphocytes* | (0.3-20) · 10⁹/L | 0.99 |
| Monocytes* | (0.1-6.6) · 10⁹/L | 0.96 |
| Granulocytes* | (1.0-31) · 10⁹/L | 0.97 |
| Thrombocytes | (8-1,170) · 10⁹/L | 0.90 |
| Hemoglobin | (2.9-10.7) mmol/L | 0.97 |

*subpopulation of leukocytes

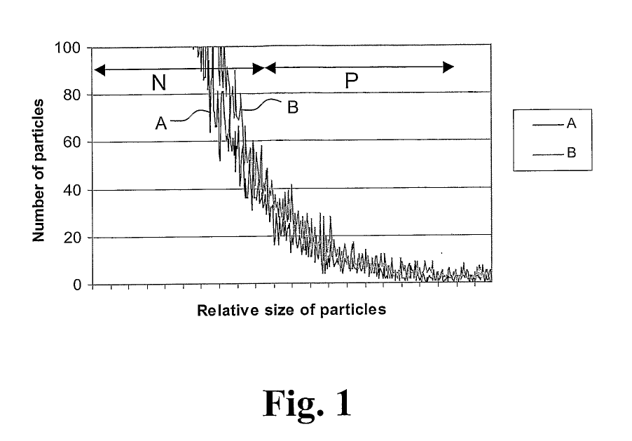

FIG. 1 shows a histogram comparing number of particles counted in two samples of the same blood. The blood samples were prepared with a reagent according to the present invention and a known single reagent system (0.704 gram dodecyltrimethylammonium chloride, 0.150 gram hexadecyltrimethylammonium bromide and 1.796 gram of 1,2,4-triazole dissolved in 360 ml of Diluide III Diff.((J. T. Baker prod. no. 3963)) and 116 ml of de-ionized water, respectively. The two prepared blood samples were analyzed using a cartridge as described below. Special electronic equipment was employed to record the cell size in a large number of intervals.

In FIG. 1 graph A shows the test result obtained with the blood sample with the known reagent, while B shows the test result obtained with the blood sample with the reagent according to the invention. P designates the thrombocytes size range, which ranges from about 1.5 μm to about 4 μm. N designates the background noise. It is noted that the counted number of thrombocytes using the reagent according to the present invention is larger than the counted number of thrombocytes using the known reagent, which is believed to be caused by a more gentle preparation of thrombocytes by the reagent according to the invention. Thus an improved preservation of thrombocytes, and thus a more precise and accurate analysis of the blood sample is obtained.

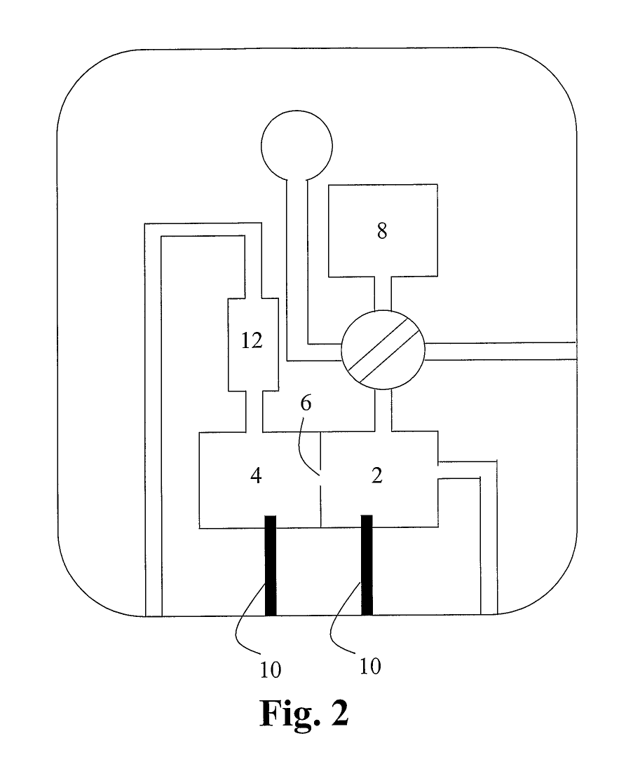

FIG. 2 schematically illustrates a disposable cartridge 1 holding a reagent according to the present invention. The cartridge is utilized in an apparatus for analysis of blood sample content that comprises a docking station and a reader. The cartridge comprises a housing with a mixing chamber 2 and a collection chamber 4 separated by a wall containing an orifice 6 for passage of the cells between the mixing chamber and the collection chamber. The reagent is accommodated in a holding chamber 8, and cell characterization means 10 are provided for characterizing cells passing through the orifice. Furthermore, the cartridge comprises a volume-metering chamber 12 connected to the collection chamber. A part of the cartridge, e.g. the mixing chamber 2, the collection chamber 4 and/or the volume-metering chamber 12, may be adapted for spectrophotometric quantification of hemoglobin content. In the shown embodiment, the volume-metering chamber 12 is adapted for spectrophotometric quantification of hemoglobin content in the blood.

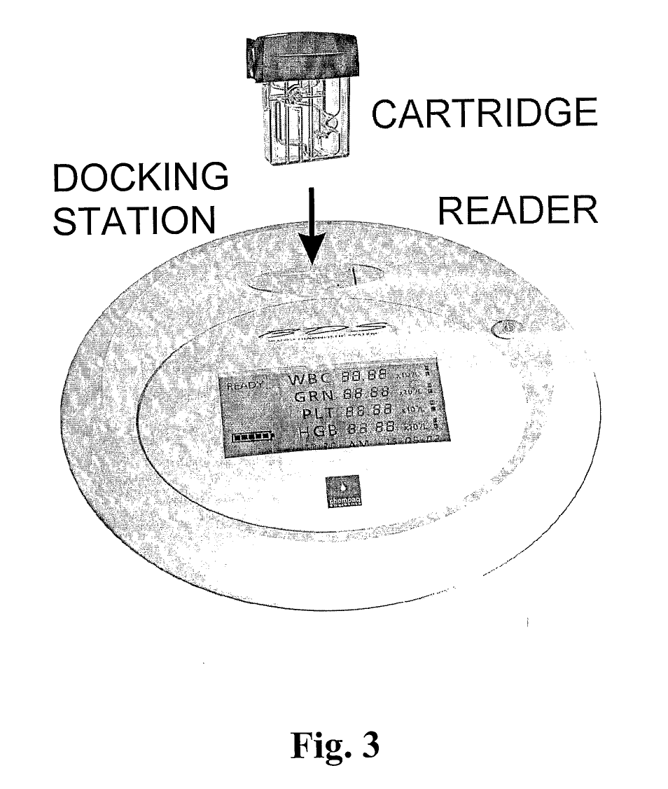

The apparatus for analysis of blood sample content is illustrated in FIG. 3 and comprises a docking station and a reader for removably receiving the cartridge, the docking station comprising connectors (not shown), e.g. electrical and/or fluid connectors, for operational connection with the cell characterization means when the cartridge is received in the docking station.

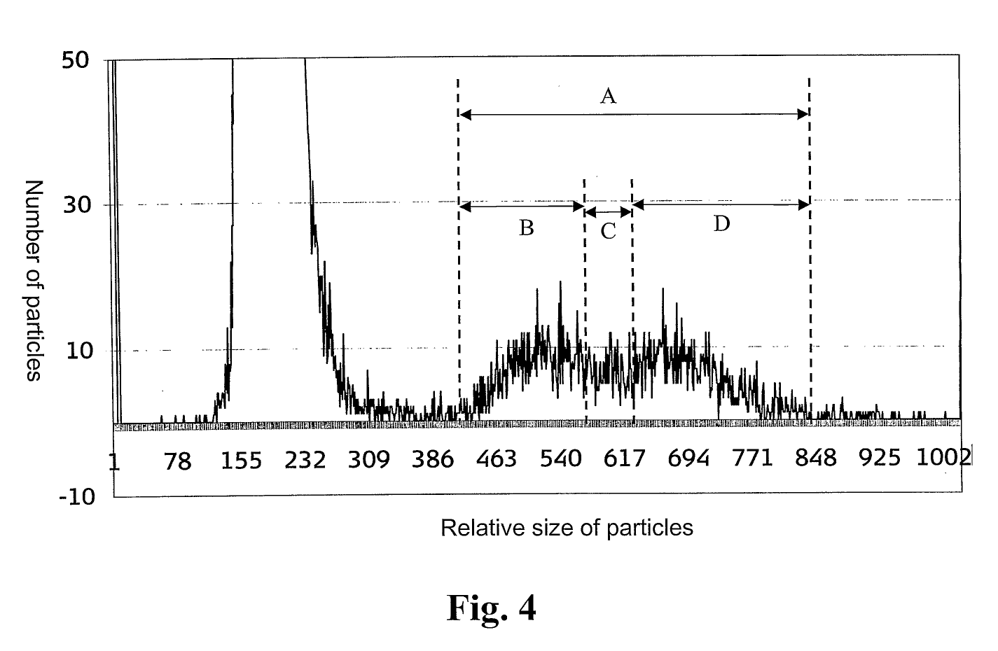

FIG. 4 shows a favorable histogram for counting thrombocytes, leukocytes and leukocyte subpopulations, obtained by using the reagent described in Table 2. A designates the part of the histogram that corresponds to leukocytes, B, C and D corresponds to lymphocytes, monocytes and granulocytes, respectively.

### Example 2

### Reagent According to the Invention

A reagent was prepared with a composition according to Table 4. The final osmolality of the prepared reagent was 330 mosmol/L.

TABLE 4

| Compound | Concentration | |
|---|---|---|
| Imidazole | 50 | mmol/L |
| Dodecyltrimethylammonium chloride | 5.6 | mmol/L |
| Hexadecyltrimethylammonium bromide | 0.9 | mmol/L |
| 1,3-dimethylurea | 17 | mmol/L |
| ADA (N-2-(acetamido)iminodiacetic acid) | 5.3 | mmol/L |
| Na₂SO₄ | 70 | mmol/L |
| NaCl | 41 | mmol/L |
| H₂SO₄, diluted | 7 | mL (to obtain |
| 1/9 (vol./vol.) with water. | | pH 7.1) |

| | |
|---|---|
| De-ionized water | To obtain 100 mL |

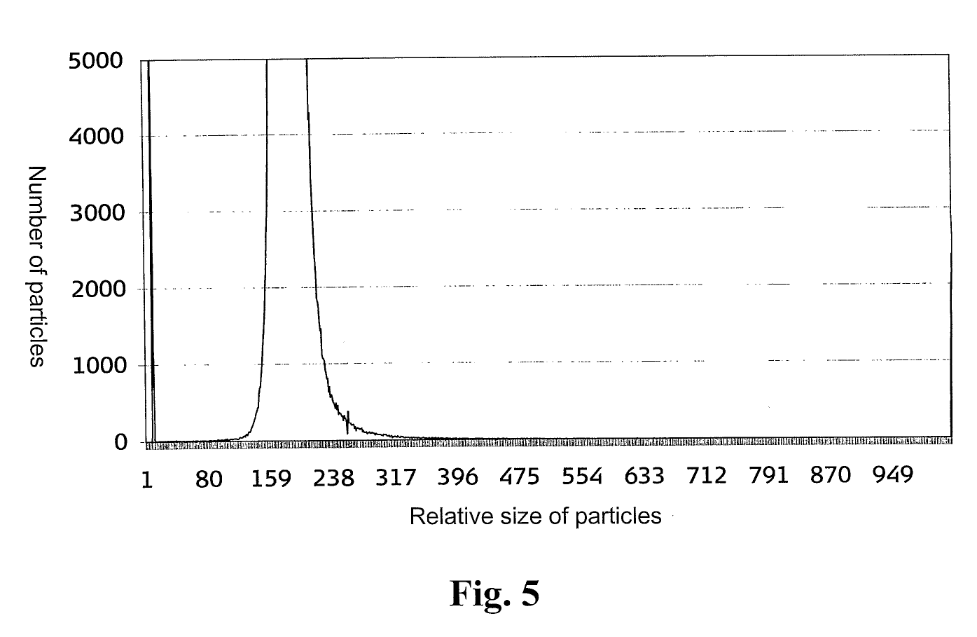

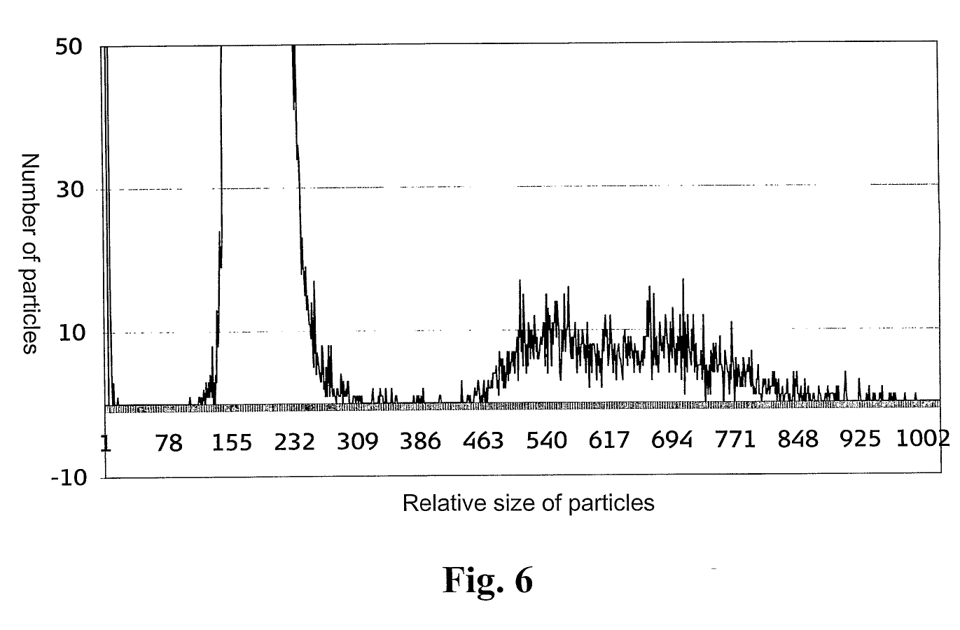

Test results obtained by using a reagent according to the invention, as described in table 4, to determine the content of the various blood cell can be seen in FIG. 5 showing a favorable histogram for counting thrombocytes, and in FIG. 6 showing a favorable histogram for counting leukocytes and leukocyte subpopulations. FIG. 5 and FIG. 6 are the one and same histogram, except that the Y-axis is different; to more clearly show the different cell-subtypes.

### Example 3

### Enhancement of the Capillary Effect by Use of the Inventive Reagent as a Coating

Surprisingly, a coating comprising the reagent according to the present invention has shown to enhance the capillary effect of a hydrophobic tube or tube-like device. The reagent is coated onto a surface by evaporation of the reagent on the surface to be coated. The effect can be explained by taking the amphiphilic nature of the reagent components into consideration. The hydrophobic molecular parts of these adhere to the hydrophobic surface of the tube resulting in an orientation of the hydrophilic molecular parts of the reagent components out from the tube surface. The inlet of a hydrophilic sample, e.g. a blood sample, will subsequently be facilitated by interaction with the hydrophilic molecular parts of oriented reagent components. The enhancement of capillary effect, i.e. increased speed of inlet of e.g. a blood sample, is therefore mainly due to the hydrophilisation of hydrophobic surfaces in the capillary tube by the reagent.

The enhanced capillary effect using a reagent according to the present invention was observed by comparing the speed of inlet of blood samples in standard capillary glass tubes (microcaps). The capillary tubes were treated with a reagent according to example 1 and comparison were made with similar treatment using a solution of 3% (weight/volume) of Brij 35 in de-ionised water—a well-known very efficient compound for enhancing hydrophilicity.

A) 10 glass microcaps with a volume of 20 μL, length 64 mm, (Drummond Inc. prod. no 1-000-0200) were filled with a reagent (as described in Example 1); and

B) another 10 glass microcaps were filled with Brij 35 (3% weight/ volume, Sigma prod. no. 228340050) in de-ionized water.

All 20 microcaps were thereafter emptied by contacting them to a paper tissue, and allowed to dry overnight at ambient temperature.

The next day the speed of inlet of microcaps without treatment were compared to the speed of inlet of microcaps treated with A) and B), respectively. See table 5.

The speed of inlet was determined by putting the end of each type of microcap into a venous (EDTA) blood sample and measuring the height of blood in each microcap after 3 seconds.

TABLE 5

| | Height of blood in microcap |
|---|---|
| Type of microcap | after 3 seconds* |
| Untreated | 30 mm |
| A) Reagent | 42 mm |
| B) Brij 35 | 43 mm |

*Average of 10 runs.

Conclusion:

The microcaps treated with the reagent of Example 1 were filled with blood with a speed in the same order as those filled with Brij 35.

1-32. (canceled)

33. A reagent comprising
a hemoglobin-stabilizing compound selected from the group consisting of imidazole, imidazole derivatives, and combinations thereof;
at least two quaternary ammonium salts;
1,3-dimethylurea and/or salts thereof; and
an organic buffer.

34. A reagent according to claim 33, wherein the concentration of the hemoglobin-stabilizing compound selected from the group consisting of imidazole, imidazole derivatives, and combinations thereof in the reagent is in the range from about 10 mmol/L to about 100 mmol/L, such as from about 25 mmol/L to about 75 mmol/L, from about 40 mmol/L to about 60 mmol/L, and preferably about 50 mmol/L.

35. A reagent according to claim 33, where the hemoglobin-stabilizing compound is imidazole in a concentration of about 50 mmol/L.

36. A reagent according to claim 33, wherein the at least two quaternary ammonium salts comprise a combination of dodecyltrimethylammonium chloride and hexadecyltrimethylammonium bromide.

37. A reagent according to claim 33, wherein the total concentration of the at least two quaternary ammonium salts in the reagent is in the range from about 1.0 mmol/L to about 10 mmol/L, such as from about 3.0 mmol/L to about 9.0 mmol/L, from about 4.5 mmol/L to about 8.5 mmol/L, and preferably about 6.5 mmol/L.

38. A reagent according to claim 33, wherein the at least two quaternary ammonium salts is a combination of dodecyltrimethylammonium chloride and hexadecyltrimethylammonium bromide in concentrations of about 5.6 mmol/L and about 0.9 mmol/L, respectively.

39. A reagent according to claim 33, wherein the concentration of 1,3-dimethylurea and/or salts thereof in the reagent is in the range from about 10 mmol/L to about 25 mmol/L, such as from about 12 mmol/L to about 23 mmol/L, from about 14 mmol/L to about 20 mmol/L, from about 16 mmol/L to about 18 mmol/L, and preferably about 17 mmol/L.

40. A reagent according to claim 33, wherein the 1,3-dimethylurea and/or salts thereof is 1,3-dimethylurea in a concentration of about 17 mmol/L.

41. A reagent according to claim 33, wherein the reagent further comprises procaine, procaine hydrochloride or other salts thereof.

42. A reagent according to claim 41, wherein the concentration of procaine hydrochloride in the reagent is in the range from about 0.2 mmol/L to about 1.6 mmol/L, such as from about 0.4 mmol/L to about 1.4 mmol/L, from about 0.6 mmol/L to about 1.2 mmol/L, from about 0.8 mmol/L to about 1.0 mmol/L, and preferably about 0.9 mmol/L.

43. A reagent according to claim 33, wherein the organic buffer is a compound such as ADA (N-2-(acetamido)iminodiacetic acid), HEPES (2-(4(2-hydroxyethyl)-1-piperazine)ethanesulphonic acid), MOPS (3-(N-morpholino)propane sulphonic acid), PIPES (piperazine-N,N′-bis(2-ethane sulphonic acid) or combinations thereof, with a concentration in the range from about 2 mmol/L to about 8 mmol/L, such as from about 4 mmol/L to about 6 mmol/L, from about 5 mmol/L to about 5.6 mmol/L, and preferably about 5.3 mmol/L.

44. A reagent according to claim 33, wherein the organic buffer is ADA (N-2-(acetamido)iminodiacetic acid) with a concentration in the range from about, 2 mmol/L to about 8 mmol/L, such as from about 4 mmol/L to about 6 mmol/L, from about 5.0 mmol/L to about 5.6 mmol/L, and preferably about 5.3 mmol/L.

45. A reagent according to claim 33, wherein the organic buffer is ADA (N-2-(acetamido)iminodiacetic acid) with a concentration of about 5.3 mmol/L.

46. A reagent according to claim 33, wherein the osmolality of the reagent is adjusted with at least one inorganic salt, such as sodium chloride, and/or sodium sulphate.

47. A reagent according to claim 33, wherein the osmolality of the reagent is adjusted with sodium chloride and sodium sulphate.

48. A reagent according to claim 46, wherein the osmolality of the reagent is in the range from about 200 mosmol/L to about 400 mosmol/L, such as from about 250 mosmol/L to about 380 mosmol/L, from about 300 mosmol/L to about 350 mosmol/L, and preferably about 330 mosmol/L

49. A reagent according to claim 33, wherein the pH of the reagent is adjusted with an acid, and the pH of the reagent is in the range from about 6 to about 8, such as from about 6.5 to about 7.5, and preferably about 7.1.

50. A reagent according to claim 33, comprising imidazole in a concentration of about 50 mmol/L; dodecyltrimethylammonium chloride in a concentration of about 5.6 mmol/L; hexadecyltrimethylammonium bromide in a concentration of about 0.9 mmol/L; 1,3-dimethylurea in a concentration of about 17 mmol/L; ADA (N-2-(acetamido)iminodiacetic acid) in a concentration of 5.3 mmol/L; procaine hydrochloride in a concentration of 0.9 mmol/L; and de-ionized water; wherein pH is adjusted to 7.1 with diluted sulfuric acid and the osmolality is adjusted with sodium chloride and sodium sulphate.

51. A coating comprising a reagent as defined in claim 33.

52. A method of determining the content of blood cells in a blood sample, the method comprising the steps of:
i) Mixing a blood sample with a reagent as defined in claim 33, and
ii) Analyzing the content of the blood sample by employment of the Coulter principle.

53. A method according to claim 52, wherein the thrombocyte content is determined by step ii).

54. A method according to claim 52, wherein the leukocyte content is determined by step ii).

55. A method according to claim 52, wherein one or more leukocyte subpopulations such as, e.g., the lymphocytes, monocytes and/or granulocytes are determined by step ii).

56. A method according to claim 52, wherein the method further comprises
iii) Analyzing the content of the blood sample by spectrophotometric quantification of the hemoglobin content.

57. A method according to claim 52, wherein the blood:reagent-ratio by volume is in the range from about 1:10,000 to about 1:200, such as from about 1:1,000 to about 1:300, from about 1:600 to about 1:400, from about 1:550 to about 1:450, and preferably about 1:500.

58. A method according to claim 52, wherein
step i) is performed in a cartridge suitable for use in an apparatus for enumeration of cells in a blood sample, in which cartridge the reagent is present in a holding chamber that is connected with a mixing chamber in which the reagent is mixed with the blood sample; and
step ii) is performed by subsequent passage of the blood sample comprising cells through an orifice positioned between the mixing chamber and a collection chamber, whereby the content of the blood is analyzed by employment of the Coulter principle by cell characterization means.

59. A method according to claim 52, wherein step iii) is performed in a volume metering chamber connected to the collection chamber and adapted to spectrophotometric quantification.

60. Use of a reagent as defined in claim 33, for determination of the content of thrombocytes in a blood sample.

61. Use according to claim 60, for simultaneous determination of the content of leukocytes in the same blood sample.

62. Use according to claim 60, for simultaneous determination of one or more leukocyte subpopulations in the same blood sample.

63. Use according to claim 62, wherein the leukocyte subpopulation includes lymphocytes, monocytes and/or granulocytes.

64. Use according to claim 60, for determination of the hemoglobin content in the same blood sample.
